(19) United States
(12) Patent Application Publication
(10) Pub. No.: US 2015/0289839 A1
(43) Pub. Date: Oct. 15, 2015

(54) ULTRASOUND IMAGING APPARATUS AND ULTRASOUND IMAGE DISPLAY METHOD

(71) Applicant: KONICA MINOLTA, INC., Tokyo (JP)

(72) Inventors: Masahiro SAITO, Yokohama-shi (JP); Akihiro KAWABATA, Tokyo (JP)

(73) Assignee: KONICA MINOLTA, INC., Tokyo (JP)

(21) Appl. No.: 14/676,342

(22) Filed: Apr. 1, 2015

(30) Foreign Application Priority Data
Apr. 9, 2014 (JP) 2014-080334

(51) Int. Cl. A61B 8/08; A61B 8/00; A61B 8/14

(52) U.S. Cl. CPC A61B 8/0841; A61B 8/14; A61B 8/461; A61B 8/5215

(57) ABSTRACT

An ultrasound imaging apparatus, including: an ultrasound probe for transmitting, in response to drive signals, ultrasound waves to a subject with a puncture needle inserted therein, and outputting reception signals obtained by receiving reflected ultrasound waves from the subject; an image generating unit for generating an ultrasound image based on the reception signals; a selecting unit for selecting a range to emphasize an image of a portion of the puncture needle in the ultrasound image; a needle location detecting unit for detecting the image of the portion of the puncture needle based on the reception signals or the ultrasound image; an emphasizing unit for emphasizing the detected image of the portion of the puncture needle within the selected range; and a display unit for displaying an ultrasound image in which the image of the portion of the puncture needle is emphasized.

### BACKGROUND OF THE INVENTION

1. Field of the Invention

The present invention relates to an ultrasound imaging apparatus and a method of displaying ultrasound images.

2. Description of Related Art

Biopsy has been performed that involves insertion of a puncture needle into a living body for sampling a body tissue or bodily fluid to be used for diagnosis. In order to avoid insertion of the puncture needle into a wrong site during sampling of a certain tissue from a living body in biopsy, the puncture needle is attached to an ultrasound probe that includes a puncture adapter or guide. An operator, such as a medical doctor, displays ultrasound images of the internal body on a monitor screen with the ultrasound probe, and determines a puncture site through observation of the ultrasound images to perform paracentesis.

During the paracentesis, the operator inserts the puncture needle through a predetermined insertion route while observing the ultrasound images in order to certainly reach the target site with the puncture needle.

During such paracentesis, the operator needs to operate the puncture needle such that the needle reaches the target site, extracts unwanted fluid from the site, and/or injects fluid into the site. It is thus important that the operator can certainly and adequately observe the puncture needle, especially the tip of the needle, on the monitor.

In view of such a situation, there is disclosed, for example, a technique that involves extraction of a region which has high-brightness and experienced a significant change in brightness from a displayed ultrasound image as the tip of the puncture needle, and coloring of the extracted region to emphasize the region in the latest ultrasound image (for example, see Japanese Patent Application Laid Open Publication No. 2000-107178).

Puncture adapters and guides are designed such that the track of the tip of a puncture needle lies within a predetermined distance from (within an error range of) a predetermined insertion route after the puncture needle is actually inserted. The location of the tip of the puncture needle, therefore, is confined within the error range if a puncture adapter or guide is used. Conventionally, detection and emphasis of the puncture needle, however, have been generally performed on an entire ultrasound image, occasionally leading to erroneous emphasis of a distant location from the insertion route as the puncture needle under the influence of body motion or noise. As a result, the operator cannot readily recognize the location of the puncture needle, leading to confusion.

If the operator inserts the puncture needle freehand without the puncture adapter or guide, the insertion route may significantly stray from the predetermined insertion route depending on the skill of the operator. In this case, if an emphasis range (detection range) for the location of the tip of the puncture needle is limited, the location of the tip of the puncture needle is not emphasized, and the operator cannot determine the location of the puncture needle.

### SUMMARY OF THE INVENTION

An object of the present invention is to enable the operator to readily determine the location of the tip of the puncture needle.

In order to achieve the above object, according to one aspect of the present invention, there is provided an ultrasound imaging apparatus, including: an ultrasound probe for transmitting, in response to drive signals, ultrasound waves to a subject with a puncture needle inserted therein, and outputting reception signals obtained by receiving reflected ultrasound waves from the subject; an image generating unit for generating an ultrasound image based on the reception signals output from the ultrasound probe; a selecting unit for selecting a range to emphasize an image of a portion of the puncture needle in the ultrasound image; a needle location detecting unit for detecting the image of the portion of the puncture needle based on the reception signals or the ultrasound image; an emphasizing unit for emphasizing the image of the portion of the puncture needle detected by the needle location detecting unit within the range selected by the selecting unit; and a display unit for displaying an ultrasound image in which the image of the portion of the puncture needle is emphasized.

Preferably, the selecting unit selects a predetermined area or the entire ultrasound image as the range to emphasize the image of the portion of the puncture needle, the predetermined area being around a predetermined insertion route for the puncture needle in the ultrasound image.

Preferably, the ultrasound imaging apparatus further includes a switching unit for switching between activation and deactivation of emphasis of the image of the portion of the puncture needle in the ultrasound image.

Preferably, the ultrasound imaging apparatus further includes a first setup unit for setting an image lag factor for generating a lag image, the image lag factor for representing movement of the puncture needle, wherein the emphasizing unit generates, by the image lag factor, the lag image in which an image region corresponding to the image of the portion of the puncture needle is emphasized.

Preferably, the ultrasound imaging apparatus further includes a brightness conversion map selecting unit for selecting a brightness conversion map desired by an operator from a plurality of brightness conversion maps defining a relation between input brightness and output brightness of the ultrasound image, wherein the emphasizing unit adds a predetermined value to brightness of an image region corresponding to the image of the portion of the puncture needle in the ultrasound image and performs brightness conversion with the brightness conversion map selected by the brightness conversion map selecting unit to emphasize the image region corresponding to the image of the portion of the puncture needle.

Preferably, the ultrasound imaging apparatus further includes a second setup unit for setting an emphasis level of the image of the portion of the puncture needle, wherein the predetermined value added to the brightness of the image region corresponding to the image of the portion of the puncture needle is a value corresponding to the emphasis level set by the second setup unit.

Preferably, the ultrasound imaging apparatus further includes a brightness conversion map selecting unit for selecting a brightness conversion map desired by an operator from a plurality of brightness conversion maps defining a relation between input brightness and output brightness of the ultrasound image, wherein the emphasizing unit performs gamma conversion on brightness of the ultrasound image with the brightness conversion map selected by the brightness conversion map selecting unit and thereafter adds a predetermined color component to an image region corresponding to the image of the portion of the puncture needle to emphasize the image region corresponding to the image of the portion of the puncture needle.

Preferably, the portion of the puncture needle is a tip of the puncture needle.

According to another aspect of the present invention, there is provided an ultrasound image display method, including: generating an ultrasound image based on reception signals output from an ultrasound probe which transmits, in response to drive signals, ultrasound waves to a subject with a puncture needle inserted therein and outputs the reception signals obtained by receiving reflected ultrasound waves from the subject; selecting a range to emphasize an image of a portion of the puncture needle in the ultrasound image;

detecting the image of the portion of the puncture needle based on a plurality of frames of ultrasound images; emphasizing the detected image of the portion of the puncture needle within the selected range; and displaying an ultrasound image in which the image of the portion of the puncture needle is emphasized.

Preferably, the portion of the puncture needle is a tip of the puncture needle.

In the present invention, the operator can readily determine the location of the tip of the puncture needle.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is a diagram illustrating an external configuration of an ultrasound imaging apparatus;

FIG. 2 is a diagram illustrating an exemplary puncture adapter attached to an ultrasound probe;

FIG. 3 is a block diagram illustrating a schematic configuration of the ultrasound imaging apparatus;

FIG. 4 is a block diagram illustrating a functional configuration of an image processing unit;

FIG. 5A is a diagram illustrating a first screen example for explaining screen operations for shift to a puncture mode;

FIG. 5B is a diagram illustrating a second screen example for explaining screen operations for shift to a puncture mode;

FIG. 6 is a diagram illustrating an exemplary display of a puncture guideline and error lines;

FIG. 7 is a diagram illustrating screen operations to display an image with the emphasized puncture needle;

FIG. 8 is a diagram illustrating an exemplary options window to set various parameters for displaying an image with the emphasized puncture needle;

FIG. 9A is a diagram illustrating a selectable range for displaying an image with the emphasized puncture needle;

FIG. 9B is a diagram illustrating another selectable range for displaying an image with the emphasized puncture needle;

FIG. 10 is a flow chart illustrating an ultrasound image generating display process performed by the control unit of FIG. 3; and

FIG. 11 is a diagram illustrating an exemplary gamma curve.

### DETAILED DESCRIPTION OF THE PREFERRED EMBODIMENT

Embodiments of an ultrasound imaging apparatus according to the present invention will now be described with reference to the drawings. These exemplary embodiments should, however, not be construed to limit the scope of the invention.

### [Configuration of Ultrasound Imaging Apparatus]

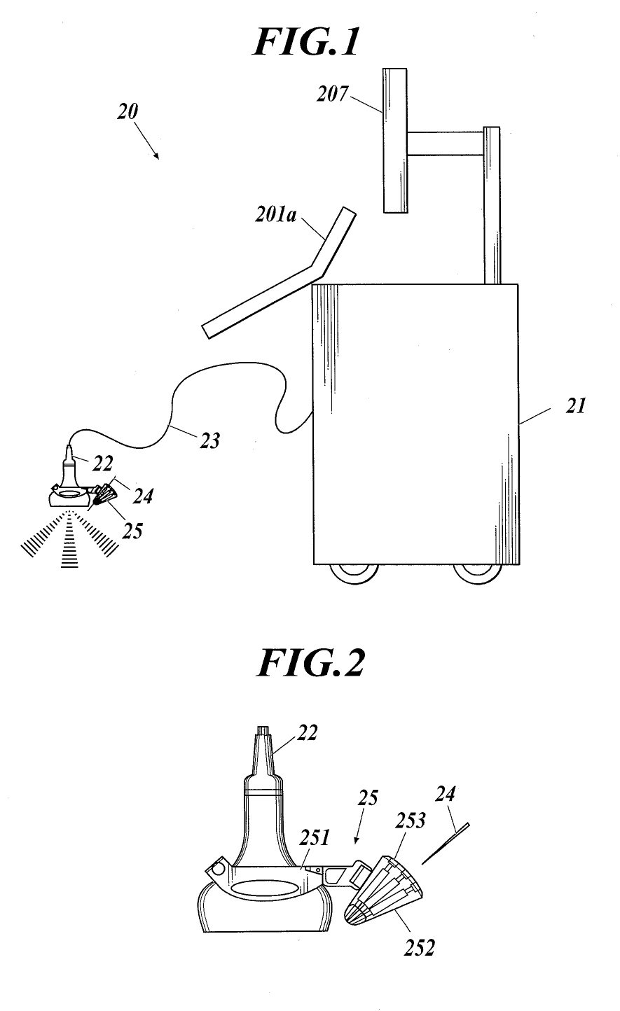

With reference to FIG. 1, an ultrasound imaging apparatus 20 according to the embodiment includes a main unit 21 and an ultrasound probe 22. The ultrasound probe 22 transmits ultrasound waves to a subject, such as a living body (not shown), and receives reflected waves (reflected ultrasound waves: echo) of the ultrasound waves that are reflected from the subject. The ultrasound probe 22 is connected to the main unit 21 with a cable 23. The main unit 21 transmits electrical drive signals to the ultrasound probe 22 to cause the ultrasound probe 22 to transmit the ultrasound waves to the subject, and creates ultrasound images on internal conditions of the subject based on electrical signals called reception signals generated in the ultrasound probe 22 in response to the ultrasound waves reflected from the interior of the body of the subject.

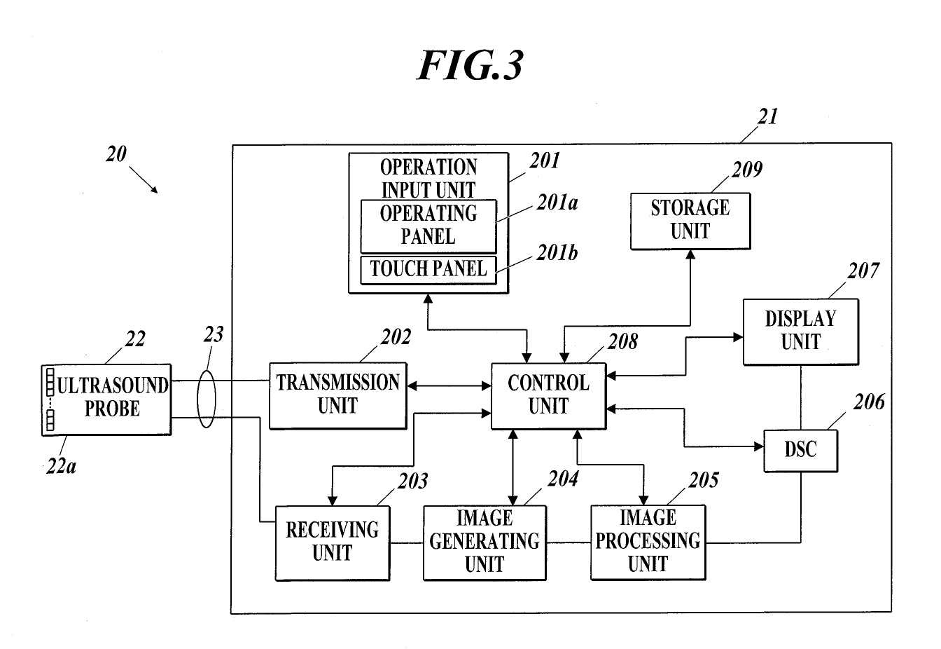

The ultrasound probe 22 includes multiple transducers 22a made of a piezoelectric element (see FIG. 3), and the transducers 22a are disposed in a one-dimensional array in the horizontal direction, for example. In the embodiment, the ultrasound probe 22 includes 192 transducers 22a, for example. The transducers 22a may be disposed in a two-dimensional array. Furthermore, the number of transducers 22a may be any appropriate number. The ultrasound probe 22 may be operated electronically or mechanically. In addition, the ultrasound probe 22 may be operated by any of the linear, sector, and convex modes.

The ultrasound probe 22 is provided, on its side, with a puncture adapter 25 for guiding a puncture needle 24 for insertion to the subject in the horizontal direction. An exemplary puncture adapter 25 attached to the ultrasound probe 22 is shown in FIG. 2. As shown in FIG. 2, the puncture adapter 25 includes a holder 251 and a puncture needle guide 252.

The holder 251 is a member for attaching the puncture adapter 25 to the ultrasound probe 22 so as to be removable. It is preferable that the holder 251 is attached to the ultrasound probe 22 with a film sheath therebetween to prevent infection caused by bodily fluid and blood.

The puncture needle guide 252 has a fan shape, for example, as shown in FIG. 2, and is attached to the holder 251 so as to be removable. The puncture needle guide 252 includes multiple needle insertion grooves 253 for insertion of the puncture needle 24. As shown in FIG. 2, these needle insertion grooves 253 have different angles of inclination from one another with respect to a contact surface of the ultrasound probe 22 with the subject. In other words, the puncture needle 24 is inserted to the subject through the plurality of needle insertion grooves 253 at different puncture (insertion) angles.

In the embodiment, instead of the puncture adapter 25, the ultrasound probe 22 may include, for example, a guide groove for guiding the puncture angle of the puncture needle 24.

As shown in FIG. 3, the main unit 21 includes, for example, an operation input unit 201, a transmission unit 202, a receiving unit 203, an image generating unit 204, an image processing unit 205, a digital scan converter (DSC) 206, a display unit 207, a control unit 208, and a storage unit 209.

The operation input unit 201 includes an operating panel 201a and a touch panel 201b. The operating panel 201a includes, for example, various switches, buttons, a trackball, a mouth, and a keyboard with which the operator inputs a command for starting a diagnosis or data, such as personal information of the subject, and performs a freeze operation, and outputs operating signals to the control unit 208. The touch panel 201b is integrated with a display of the display unit 207, and detects an operation performed with a finger or the like on the display of the display unit 207 and outputs information on coordinates of the operated position to the control unit 208.

The transmission unit 202 is a circuit that transmits electrical drive signals to the ultrasound probe 22 through a cable 23 under the control of the control unit 208 to cause the ultrasound probe 22 to generate the ultrasound waves. Furthermore, the transmission unit 202 includes, for example, a clock generating circuit, a delay circuit, and a pulse generating circuit. The clock generating circuit generates clock signals to determine the transmission timing and transmission frequency for the drive signals. The delay circuit establishes a time delay for the transmission timing of the drive signals for the channel corresponding to each of the transducers 22a and delays the transmission of the drive signals by the established time delay to focus the transmitted beams of the ultrasound waves. The pulse generating circuit generates pulse drive signals at a predetermined frequency.

The transmission unit 202 in the above configuration sequentially switches the multiple transducers 22a to supply the drive signals by a predetermined number for every transmission and reception of the ultrasound waves, and transmits the drive signals to the selected multiple transducers 22a to output the ultrasound waves for scanning under the control of the control unit 208.

The receiving unit 203 receives electrical reception signals from the ultrasound probe 22 through the cable 23 under the control of the control unit 208. The receiving unit 203 includes, for example, an amplifier, an A/D converter circuit, and a phasing adder circuit. The amplifier is a circuitry for amplifying the reception signals by a predetermined multiplication factor for the channel corresponding to each of the transducers 22a. The A/D converter circuit converts the amplified analog reception signals to digital signals (A/D conversion). The phasing adder circuit phases the digital reception signals by applying the time delay to the channel corresponding to each of the transducers 22a and adding the resulting signals (phasing addition) to generate sound ray data.

The image generating unit 204 performs envelope demodulation and logarithmic amplification on the sound ray data from the receiving unit 203 to adjust the gain and converts the adjusted gain into brightness to generate B-mode images. In other words, the B-mode images represent the intensity of the reception signals in the form of brightness. The B-mode images generated by the image generating unit 204 are output to the image processing unit 205.

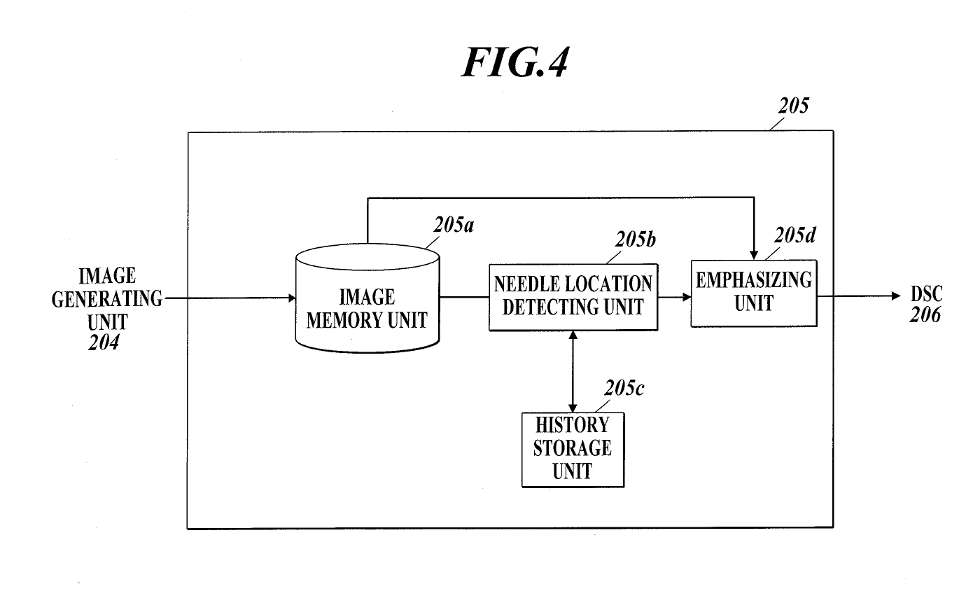

The image processing unit 205 stores the B-mode images output from the image generating unit 204 frame by frame in an image memory unit 205a (see FIG. 4). The B-mode image as a frame is referred to as an ultrasound image, or a frame image. The image processing unit 205 appropriately reads the ultrasound images stored in the image memory unit 205a to output them to the DSC 206.

Furthermore, as shown in FIG. 4, the image processing unit 205 includes the image memory unit 205a, a needle location detecting unit 205b, a history storage unit 205c, and an emphasizing unit 205d.

The image memory unit 205a consists of semiconductor memories, such as dynamic random access memories (DRAMs). The image memory unit 205a includes a high-capacity memory capable of storing data for the ultrasound images of approximately 10 seconds, and stores the ultrasound images of the most recent 10 seconds by, for example, the first-in first-out (FIFO) method.

The needle location detecting unit 205b detects a location of the tip of the puncture needle 24 based on the multiple ultrasound image frames. The tip of the puncture needle 24 is detected, for example, by obtaining difference signals C that represent differences in pixel value for every pixel between an ultrasound image A, a current frame, and an ultrasound image B, a one-frame earlier frame, and identifying a region where the obtained difference signals C corresponding to the region are equal to or greater than a predetermined value (i.e. a region that has high-brightness and a motion) as the location of the tip of the puncture needle 24. The method of detecting the location of the puncture needle for the needle location detecting unit 205b is not limited to the method involving the difference signals C, and any other approach may be used for the detection.

One approach involves detection of the location of the puncture needle by identifying the puncture needle to be inserted at an angle with respect to the probe, by steering transmission and reception for obliquely transmitting and receiving ultrasound waves or plane wave transmission and reception.

The location of the puncture needle may also be detected with the intensity of the sound ray data of the reception signals received by the receiving unit 203 or a change in acoustic data due to movement of the puncture needle.

It will be appreciated that these approaches for detecting the location of the puncture needle can detect not only the tip of the puncture needle but also a portion of the inserted puncture needle on the ultrasound image. An exemplary detection of the tip of the puncture needle will now be described.

The history storage unit 205c stores history information on the location of the tip of the puncture needle 24 detected by the needle location detecting unit 205b.

The emphasizing unit 205d performs image processing of emphasizing image regions in the current ultrasound image that correspond to the location of the tip of the puncture needle 24 detected by the needle location detecting unit 205b and the location of the tip of the puncture needle 24 read from the history storage unit 205c.

The DSC 206 converts the ultrasound image received from the image processing unit 205 into interlaced image signals and outputs the interlaced image signals to the display unit 207.

The display unit 207 may include a display, such as a liquid crystal display (LCD), a cathode-ray tube (CRT) display, an organic electroluminescent (EL) display, an inorganic EL display, and a plasma display. The display unit 207 displays the ultrasound image on the display monitor according to the image signals output from the DSC 206.

The control unit 208 includes, for example, a central processing unit (CPU), a read only memory (ROM), and an random access memory (RAM). The control unit 208 reads various processing programs, such as system programs, stored in the ROM into the RAM, and comprehensively controls the operation of each unit of the ultrasound imaging apparatus 20 according to the programs read.

The ROM consists of non-volatile memories, such as semiconductors, and stores the system programs for the ultrasound imaging apparatus 20, various processing programs executable on the system programs, and various types of data. These programs are stored in the form of computer-readable program codes, and the CPU sequentially executes operations according to the program codes.

The RAM creates a work area for temporarily storing various programs to be executed by the CPU and data corresponding to these programs.

The storage unit 209 includes a hard disk drive (HDD) and non-volatile semiconductor memories. The storage unit 209 stores various kinds of setting information for the ultrasound imaging apparatus 20 and ultrasound images designated to be stored, for example.

### [Puncture Mode]

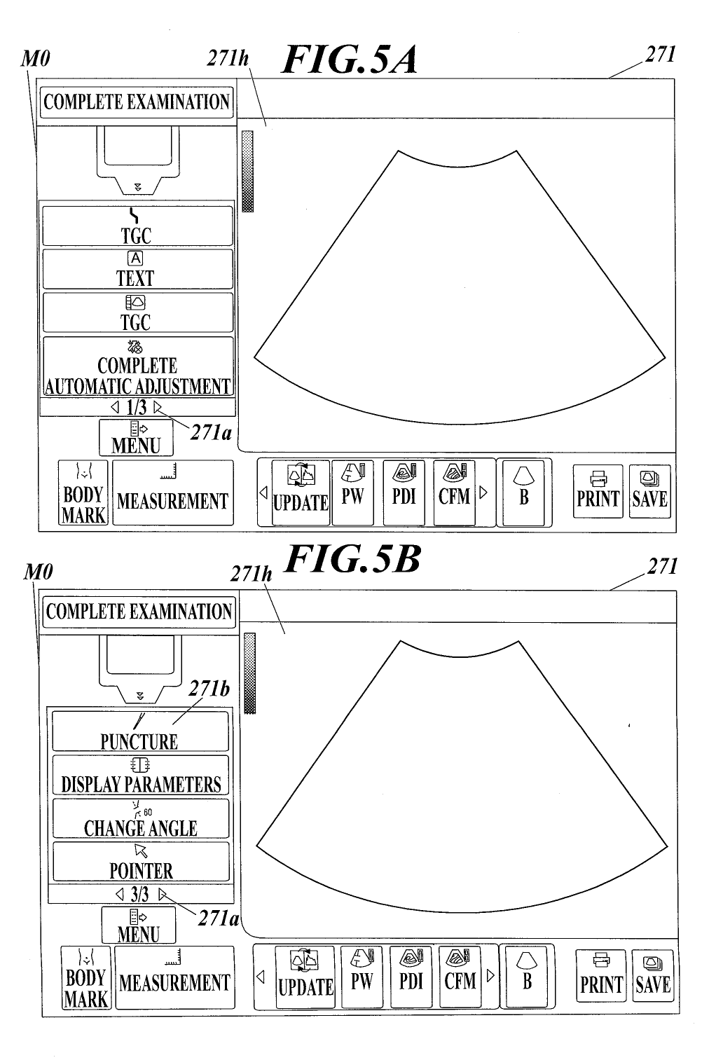

The ultrasound imaging apparatus 20 can be operated in a puncture mode for supporting the operator during the ultrasound imaging with the puncture needle 24. As shown in FIGS. 5A and 5B, for the shift to the puncture mode, the operator presses a next-page button 271a predetermined number of times in the main menu M0 on an examination window 271 appearing on the display unit 207 under the control of the control unit 208 upon startup of the ultrasound imaging apparatus 20, and then presses a puncture button 271b that appears on the menu.

The touch panel 201b detects operations with various buttons that appear on the display of the display unit 207, and the control unit 208 performs appropriate operations corresponding to the button operations (for example, transition of windows and various setting operations) in response to the operations detected by the touch panel 201b.

In the puncture mode, the operator can display a puncture guideline L1 for indicating a predetermined insertion route for the puncture needle 24 (a straight line through which the puncture needle 24 is inserted) and emphasize the tip of the inserted puncture needle 24 (needle emphasis).

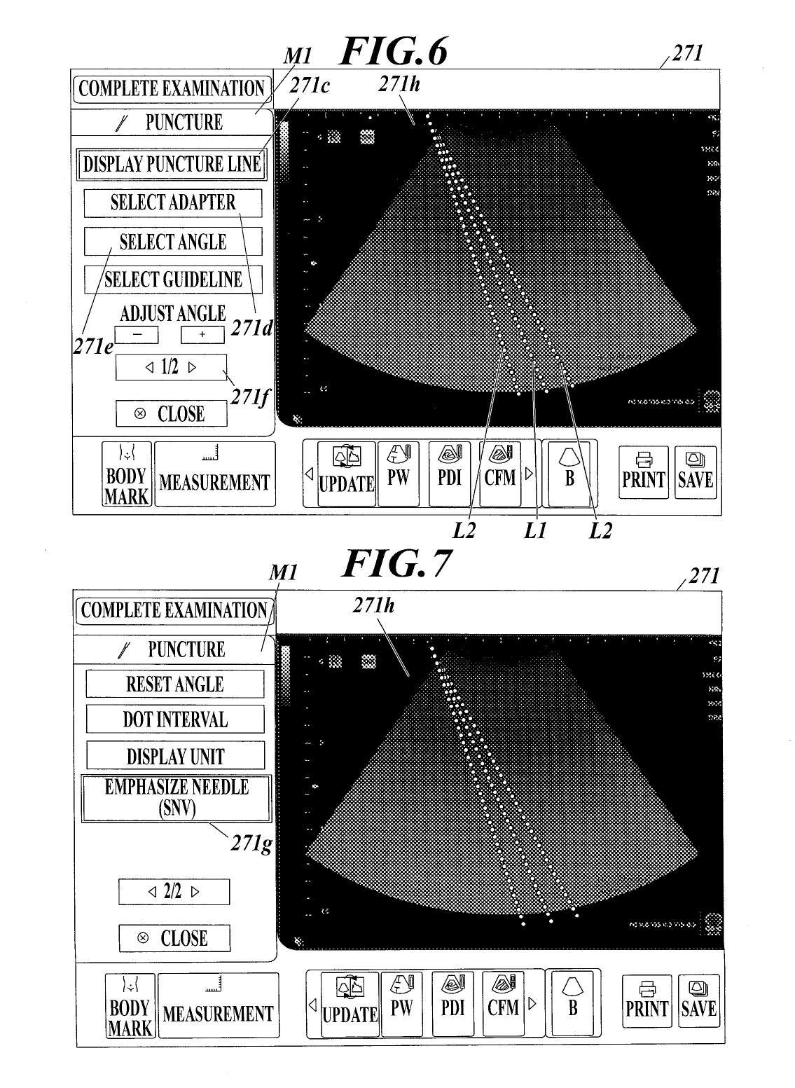

The operator displays the puncture guideline L1 by pressing a “display puncture lines” button 271c shown under a puncture menu M1 upon shifting to the puncture mode. An exemplary display of the puncture guideline L1 is illustrated in FIG. 6. In FIG. 6, the exemplary display includes error lines L2, in addition to the puncture guideline L1.

The puncture guideline L1 represents the predetermined insertion route for the puncture needle 24 that is uniquely determined based on the type of the puncture adapter 25 and the angle of the puncture needle 24 to be inserted. The operator can select the type of the puncture adapter 25 in an adapter selection window (not shown) that appears upon pressing a “select adapter” button 271d shown in FIG. 6, for example. The operator can select the angle of the puncture needle to be inserted in an angle selection window (not shown) that appears upon pressing a “select angle” button 271e shown in FIG. 6, for example. If the operator has already selected the type of the puncture adapter 25, for example, options for selection of the needle insertion groove 253 to be used appear on the angle selection window, and the operator can select the needle insertion groove 253 to be used to select the angle of the puncture needle 24 to be inserted. The error lines L2 extend from the starting point (insertion point) of the puncture guideline L1 within ±α degrees from the puncture guideline L1, where α represents an allowable error range predetermined by specifications. The puncture adapter 25 is designed such that the inserted puncture needle 24 is within ±α degrees from the puncture guideline L1. In other words, the puncture needle 24 is inserted along the approximate puncture guideline L1 within the area between the two error lines L2.

The information on the selected type of the puncture adapter 25 and the angle is stored in the storage unit 209.

If the operator represses the “display puncture lines” button 271c when the “display puncture lines” button 271c has already been pressed, the operator can disable the display of the puncture guideline L1 and the error lines L2. That is, the operator can switch on (enable) and off (disable) the display of these puncture lines by turning on and off the “display puncture lines” button 271c. The on/off setting for the display of the puncture lines is stored in the storage unit 209 by the control unit 208 according to the operation with the “display puncture lines” button 271c.

The operator can emphasize the puncture needle by pressing an “emphasize needle” button 271g (see FIG. 7) that appears upon pressing a next-page button 271f under the puncture menu M1 shown in FIG. 6. In FIG. 7, the puncture needle 24 has not been inserted. If the operator represses the “emphasize needle” button 271g when the “emphasize needle” button 271g has already been pressed, the operator can disable the emphasis of the tip of the puncture needle 24. In other words, the operator can switch on (enable) and off (disable) of the needle emphasis by turning on and off the “emphasize needle” button 271g. Information on whether the needle emphasis is on or off is stored in the storage unit 209 by the control unit 208 according to the operation with the “emphasize needle” button 271g. This means that the “emphasize needle” button 271g works as a switching unit. The “emphasize needle” button 271g on the examination window 271 enables a ready comparison between an original image before the emphasis of the puncture needle and an image after the emphasis of the puncture needle and prevents unclear observation of the target site caused by the emphasized needle blocking the target site.

According to the embodiment, the operator can set various parameters for the needle emphasis on an options window 272 for the system of the ultrasound imaging apparatus 20, which appears upon predetermined operations with the operation input unit 201.

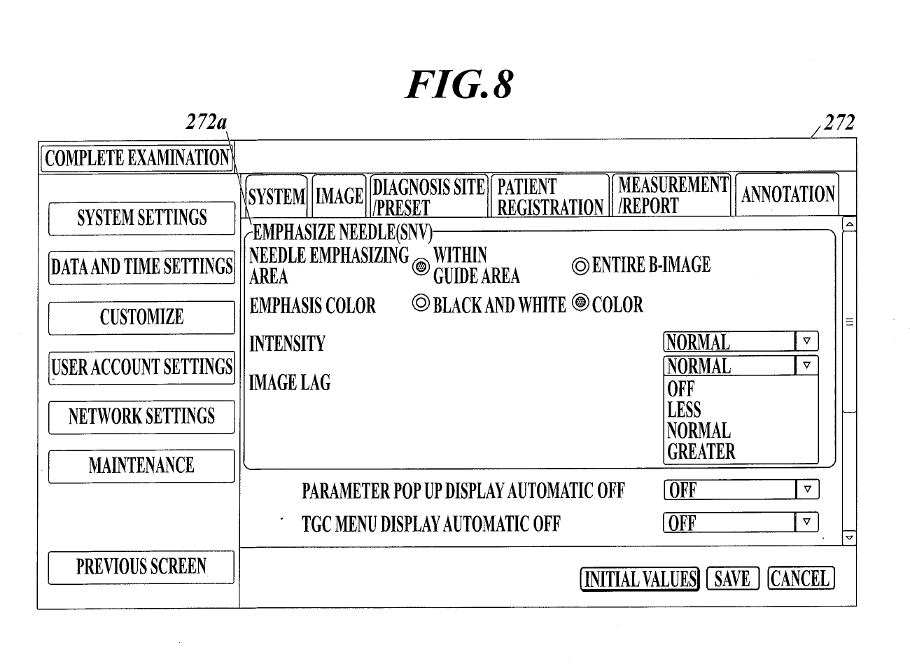

An exemplary options window 272 is illustrated in FIG. 8. The options window 272 includes a needle emphasis setting section 272a through which the operator sets various parameters for the needle emphasis. The needle emphasis setting section 272a includes options for selection of a range (needle emphasizing area), emphasis color, intensity level of emphasis, and degree of image lag for the needle emphasis in an ultrasound image.

The operator can select (set) either a shaded guide region (within the area between the two error lines L2) in FIG. 9A or the entire shaded image in FIG. 9B as the needle emphasizing range. A letter G in FIGS. 9A and 9B represents the bounds of the ultrasound image.

It is preferable to set the needle emphasizing range to be within the guide region when the operator inserts the puncture needle 24 with the puncture adapter 25. Occasionally, the location of the tip of the puncture needle 24 is erroneously detected under the influence of body motion or noise. If this erroneously detected location is emphasized, the operator cannot readily recognize the location of the puncture needle 24, leading to confusion. Limiting the needle emphasizing range within the guide region through which the puncture needle 24 is to be inserted can prevent such a situation as much as possible.

On the other hand, it is preferable to set the needle emphasizing range to be the entire image when the operator inserts the puncture needle 24 freehand without the puncture adapter 25. In this case, the operator cannot anticipate the location of the tip of the puncture needle 24. It is thus preferable to select the entire image for the needle emphasizing area such that the location of the tip can be emphasized wherever it is in the image.

The operator can select, for example, either black and white (gray) or any other color for the emphasis color.

The operator can select any intensity level of emphasis, for example, from among low, normal and high.

The operator can select any degree of image lag from among off, less, normal and greater. The degree of image lag represents the number of previous locations of the tip of the puncture needle 24 to be emphasized along with the current location in response to the movement of the puncture needle. Each of the less, normal and greater degrees corresponds to an image lag factor that is a value (number of frames) indicating the number of the previous locations of the tip of the puncture needle 24 to be displayed along with the current location. A greater degree of image lag corresponds to more number of frames (a greater factor). For example, when the operator uses a fine puncture needle 24, selection of a greater degree of image lag allows the operator to readily observe the tip of the puncture needle.

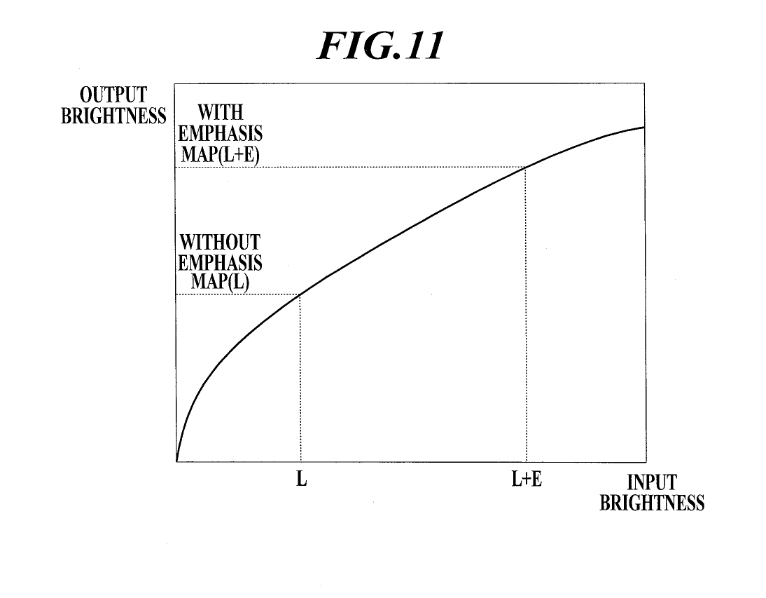

In the options window 272, the operator can select a desired gamma curve from among multiple gamma curves (see FIG. 11) with predetermined operations. The gamma curve defines a correlation between the brightness of the generated ultrasound image (input brightness) and the brightness of the displayed ultrasound image (output brightness).

The parameters set in the options window 272 are stored in the storage unit 209 by the control unit 208. The options window 272 works as a selecting unit for selecting the needle emphasizing range, a first setup unit for setting the degree of image lag, a second setup unit for setting the intensity level of emphasis for the puncture needle 24, and a brightness conversion map selecting unit for selecting a brightness conversion map such as the gamma curve.

### [Process of Generating and Displaying Ultrasound Images]

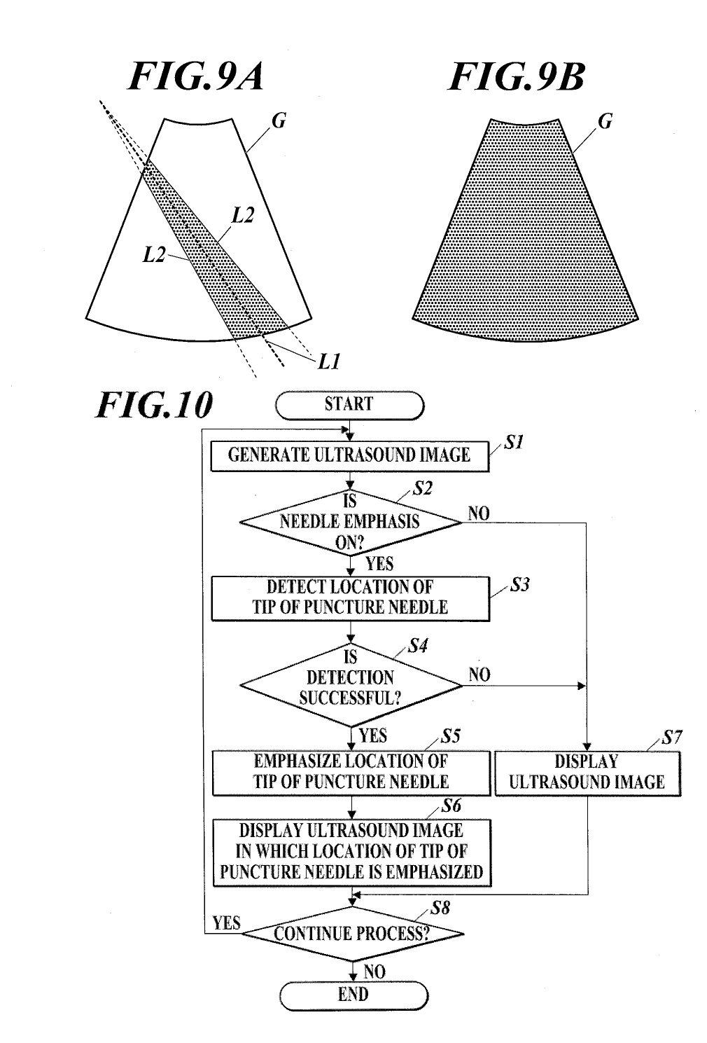

With reference to FIG. 10, an exemplary process of generating and displaying ultrasound images performed by the control unit 208 of the above-configured ultrasound imaging apparatus 20 will now be described. The process of generating and displaying ultrasound images is performed in response to predetermined operations for examination performed by the operator, such as a medical doctor or technician, in the puncture mode.

When the operator presses the ultrasound probe 22 to the body of the subject, the control unit 208 controls the transmission unit 202, the ultrasound probe 22, the receiving unit 203 and the image generating unit 204 to generate one ultrasound image frame of the body of the subject and stores the generated frame in the image memory unit 205a of the image processing unit 205 (Step S1).

The control unit 208 then refers to the storage unit 209 to determine whether the needle emphasis is on (Step S2).

If the control unit 208 determines that the needle emphasis is on (Step S2; YES), the control unit 208 causes the needle location detecting unit 205b of the image processing unit 205 to detect the location of the tip of the puncture needle 24 within the selected needle emphasizing range in the ultrasound image stored in the image memory unit 205a (Step S3).

The control unit 208 then determines whether the location of the tip of the puncture needle 24 was successfully detected by the needle location detecting unit 205b (Step S4). If the control unit 208 determines that the location of the tip of the puncture needle 24 was successfully detected (Step S4; YES), the control unit 208 causes the emphasizing unit 205d of the image processing unit 205 to perform image processing of emphasizing the location of the tip of the puncture needle 24 based on the parameters for the needle emphasis stored in the storage unit 209 (Step S5).

If the set emphasis color is black/white or high/low in brightness, the emphasizing unit 205d, as shown in FIG. 11, adds an emphasis factor E, which corresponds to the set intensity level of emphasis, to the pixel brightness L for every pixel in the image region corresponding to the current location of the tip of the puncture needle 24 detected by the needle location detecting unit 205b at the step S3 in the current ultrasound image frame generated at the step S1. The emphasizing unit 205d then reads the locations of the tip of the puncture needle 24 for the previous frames corresponding to the set degree of image lag from the history storage unit 205c. The emphasizing unit 205d also adds the emphasis factor E, which corresponds to the set intensity level of emphasis, to the pixel brightness L for every pixel in the image regions corresponding to the read locations of the tip of the puncture needle 24 in the current ultrasound image. The emphasizing unit 205d performs the gamma correction on the ultrasound image frame in which E is added to the brightness values of the pixels corresponding to the locations of the tip of the puncture needle 24 with the selected gamma curve. In other words, in the regions corresponding to the above-mentioned locations of the tip of the puncture needle 24 in the current ultrasound image frame, if the emphasis color is black and white, the emphasizing unit 205d converts the pixel brightness L+E into the brightness MAP (L+E). If the emphasis color is high/low in brightness, the emphasizing unit 205d converts the pixel brightness L+E into MAPr (L+E), MAPg (L+E) and MAPb (L+E). The emphasizing unit 205d converts pixel brightness L into MAP (L) or into MAPr (L), MAPg (L) and MAPb (L) for other regions of the current ultrasound image frame. This provides an ultrasound image (image lag) frame with an emphasized location of the tip of the puncture needle 24.

If the set emphasis color is any other color, the emphasizing unit 205d performs the gamma conversion on the current ultrasound image frame generated at the step S1 with the selected gamma curve. The emphasizing unit 205d decomposes the brightness L of each pixel in a region corresponding to the current location of the tip of the puncture needle 24 detected by the needle location detecting unit 205b at the step S3 into color components r (L), g (L) and b (L) and performs the gamma correction on each color component with the selected gamma curve. In other words, the emphasizing unit 205d converts the brightness L of each pixel into MAPr (L), MAPg (L) and MAPb (L). Similarly, the emphasizing unit 205d performs the gamma conversion on the regions corresponding to the locations of the tip of the puncture needle 24 for the previous frames, which are stored in the history storage 205c, corresponding to the set degree of image lag.

The emphasizing unit 205d then adds the color components of the emphasis color (color components of light blue color in this embodiment) to the brightness, after the gamma correction, of the pixels in the region corresponding to the location of the tip of the puncture needle 24. For example, if the ratio of the color components of the emphasis color (light blue color) is r:g:b=0:(½):1, then the red, green and blue components are MAPr (L), MAPg (L)+E/2, and MAPb (L)+E, respectively. The emphasis factor E is a predetermined value according to the set intensity level of emphasis. The emphasis factor E for the emphasis color of black/white or high/low in brightness may differ from the emphasis factor E for the full emphasis color. This provides an ultrasound image (image lag) with the emphasized location of the tip of the puncture needle 24 with the color.

For example, the location of the tip of the puncture needle 24 is emphasized by replacing the brightness thereof with certain brightness or a color, occasionally resulting in an emphasis not reflecting the gamma curve selected by the operator. The emphasizing unit 205d, however, performs the conversion with the gamma curve after adding the emphasis factor E to the brightness L of the pixels corresponding to the location of the tip of the puncture needle 24 if the emphasis color is black/white or high/low in brightness. The resulting emphasis, therefore, reflects the gamma curve selected by the operator, allowing the operator to readily observe the emphasized needle. If the emphasis color is a full color, the emphasizing unit 205d adds the color components of the emphasis color to the color components of the pixels in the region corresponding to the location of the tip of the puncture needle 24 after the conversion of the brightness L (gray component) of the ultrasound image with the gamma curve. The resulting emphasis, therefore, reflects the gamma curve selected by the operator, allowing the operator to readily observe the emphasized needle.

In this embodiment, brightness conversion with the gamma curve is described. Conversion, however, may involve various brightness conversion maps for converting brightness, such as brightness conversion with other curves than quadratic curves such as the gamma curve and conversion of brightness into pixel values in the RGB. This leads to better adaptation of the needle emphasis to various needs.

After the emphasis by the emphasizing unit 205d, the control unit 208 causes the display unit 207 (an image displaying area 271h in the examination window 271) to display the ultrasound image with the location of the puncture needle 24 being emphasized based on the ultrasound image with the emphasized location of the tip of the puncture needle 24 by the emphasizing unit 205d (Step S6), and the process proceeds to step S8.

On the other hand, if the control unit 208 determines that the needle emphasis is off (Step S2; NO) or that the detection of the location of the tip of the puncture needle by the needle location detecting unit 205b fails (Step S4; NO), the control unit 208 causes the display unit 207 (the image displaying area 271h in the examination window 271) to display the ultrasound image based on the current ultrasound image (Step S7), and the process proceeds to step S8.

In step S8, the control unit 208 determines continuation or discontinuation of the process of generating and displaying ultrasound images (Step S8). For example, the control unit 208 determines continuation or discontinuation of the process of generating and displaying ultrasound images based on input from the operation input unit 201. If the control unit 208 determines the continuation of the process (Step S8; YES), the control unit 208 returns the process to the step S1. If the control unit 208 determines the discontinuation of the process (Step S8; NO), the control unit 208 finishes the process of generating and displaying ultrasound images.

As described above, according to the ultrasound imaging apparatus 20 of the embodiment, the needle emphasis setting section 272a in the options window 272 includes options for the selection of the needle emphasizing range in which the location of the tip of the puncture needle 24 is to be emphasized in the ultrasound image. The needle location detecting unit 205b detects the locations of the tip of the puncture needle from the selected needle emphasizing range in the multiple ultrasound image frames. The emphasizing unit 205d emphasizes the image regions corresponding to the locations of the tip of the puncture needle 24 detected from the selected needle emphasizing range in the ultrasound image. The display unit 207 displays the ultrasound image in which locations of the tip of the puncture needle 24 are emphasized.

Accordingly, the operator can select the needle emphasizing range in which the puncture needle is to be emphasized depending on situations, leading to better recognition of the location of the tip of the puncture needle.

A predetermined area from a predetermined insertion route for the puncture needle 24 in an ultrasound image, for example, is selected for the needle emphasizing range. This prevents emphasis of a distant location from the insertion route through erroneous detection and allows the operator to readily recognize the location of the puncture needle 24 when the operator inserts the puncture needle 24 with the puncture adapter 25. Furthermore, the location of the tip of the puncture needle 24 is detected in a small region, leading to a reduction in processing time. The operator can select, for example, an entire image for the needle emphasizing range, allowing the location of the tip of the puncture needle 24 to be always emphasized wherever it is in the image when the operator inserts the puncture needle freehand without the puncture adapter 25.

The examination window 271 for displaying an ultrasound image includes the “emphasize needle” button 271f as the switching unit for switching between activation and deactivation of the emphasis of the tip of the puncture needle in the ultrasound image. The operator, therefore, can compare the original image before the needle emphasis with the image after the needle emphasis, preventing unclear observation of the target site caused by the emphasized needle blocking the target site.

The needle emphasis setting section 272a of the options window 272 includes options for the selection of the degree of image lag that represents the number of previous locations of the tip of the puncture needle 24 in previous frames to be emphasized along with the current location. The emphasizing unit 205d generates the image lag frame in which regions corresponding to the previous locations of the tip of the puncture needle 24 in the previous frames corresponding to the set degree of image lag are emphasized. This allows appropriate display of the location of the tip of the puncture needle 24 depending on the size of the puncture needle 24.

If the selected emphasis color is black and white, the emphasizing unit 205d adds a predetermined value to the brightness of pixels in the region corresponding to the location of the tip of the puncture needle 24 in the ultrasound image frame, performs the gamma conversion on the sum with the gamma curve selected by the operator, and emphasizes the region corresponding to the location of the tip of the puncture needle. Accordingly, the gamma curve selected by the operator is reflected and the operator can readily observe the emphasized region. The predetermined value added to the brightness of the location of the tip of the puncture needle is determined according to the intensity level of emphasis set by the operator, allowing the operator to emphasize the region with the desired intensity.

If the selected emphasis color is a full color, the emphasizing unit 205d performs the gamma conversion on the brightness of the pixels in the region corresponding to the location of the tip of the puncture needle in the ultrasound image frame with the gamma curve, then adds predetermined color components to the brightness of the pixels in the region, and emphasizes the region. This results in an emphasis reflecting the gamma curve selected by the operator, allowing the operator to readily observe the emphasized region.

While an exemplary ultrasound imaging apparatus according to the present invention has been described by reference to the embodiment, it should be understood that the invention is not limited to the disclosed embodiment.

For example, the operator can select the parameters for the puncture mode, such as the needle emphasizing range, display (enable and disable) of the puncture lines, the type of the puncture adapter, the angle of the puncture needle to be inserted, with operations in the above-described embodiment. The control unit 208, however, may automatically select the parameters based on the attachment or detachment of the puncture adapter 25. For example, when the puncture adapter 25 is attached to the ultrasound probe 22 with a connector, the control unit 208 detects the attachment or detachment of the puncture adapter 25 through this connector. If the control unit 208 detects the attachment of the puncture adapter 25 to the ultrasound probe 22, the control unit 208 selects the shaded guide region in FIG. 9A for the needle emphasizing range. If the control unit 208 detects the detachment of the puncture adapter 25 from the ultrasound probe 22, the control unit 208 selects the entire image shaded in FIG. 9B for the needle emphasizing range. The control unit 208 enables the display of the puncture lines if the puncture adapter is attached to the ultrasound probe 22, and disables the display of the puncture lines if the puncture adapter 25 is detached from the ultrasound probe 22. Furthermore, if the puncture adapter 25 is attached to the ultrasound probe 22, the control unit 208 acquires information on the type of the puncture adapter 25 and the needle insertion groove 253 with the puncture needle 24 being inserted therein through the connector and selects the type of the puncture adapter 25 and the angle of the puncture needle 24 to be inserted based on the acquired information. Such a configuration allows various optimum parameters to be automatically set on the puncture mode (for example, the needle emphasizing range and on and off of display of the puncture lines) according to the attachment or detachment of the puncture adapter 25, significantly reducing operations by the operator and improving user friendliness.

In the above embodiment, the needle location detecting unit 205b detects the location of the tip of the puncture needle 24 within the selected needle emphasizing range, and then the emphasizing unit 205d emphasizes the location of the tip of the puncture needle 24 within the range. The needle location detecting unit 205b, however, may detect the location of the tip of the puncture needle 24 from the entire ultrasound image frame, and the emphasizing unit 205d may emphasize the location of the tip of the puncture needle 24 within the needle emphasizing range.

In the above embodiment, the image lag factor represents the number of previous locations of the tip of the puncture needle 24 in previous frames to be displayed along with the current location (number of frames). The image lag factor, however, may be based on the reception signals before the conversion to ultrasound image frames and in this case, the image lag factor may be a detected change in the reception signals instead of the number of frames.

Furthermore, the detection of the location of the tip of the puncture needle 24 indicates the detection of a change in the movement of the tip of the puncture needle. In this context, the needle location detecting unit 205b may not necessarily detect the location of the tip of the puncture needle, but recognize a moving object as an approximate tip of the puncture needle 24 and detect the location of the approximate tip as the location of the tip of the puncture needle 24.

Although an exemplary configuration of the computer-readable medium for the programs according to the present invention has been disclosed with a ROM, hard disk, or semiconductor non-volatile memory in the embodiment, the configuration should not be limited to this embodiment. Another configuration of the computer-readable medium may include a portable storage medium, such as a CD-ROM. Carrier waves may be used as a medium for carrying data of the programs according to the present invention over a communication link.

Various changes can be appropriately made in the other detailed configurations and detailed operations of each of the functional units forming the ultrasound imaging apparatus.

The entire disclosure of Japanese Patent Application No. 2014-080334 filed on Apr. 9, 2014 including description, claims, drawings, and abstract are incorporated herein by reference in its entirety.

What is claimed is:

1. An ultrasound imaging apparatus, comprising:
an ultrasound probe for transmitting, in response to drive signals, ultrasound waves to a subject with a puncture needle inserted therein, and outputting reception signals obtained by receiving reflected ultrasound waves from the subject;
an image generating unit for generating an ultrasound image based on the reception signals output from the ultrasound probe;
a selecting unit for selecting a range to emphasize an image of a portion of the puncture needle in the ultrasound image;
a needle location detecting unit for detecting the image of the portion of the puncture needle based on the reception signals or the ultrasound image;
an emphasizing unit for emphasizing the image of the portion of the puncture needle detected by the needle location detecting unit within the range selected by the selecting unit; and
a display unit for displaying an ultrasound image in which the image of the portion of the puncture needle is emphasized.

2. The ultrasound imaging apparatus of claim 1, wherein the selecting unit selects a predetermined area or the entire ultrasound image as the range to emphasize the image of the portion of the puncture needle, the predetermined area being around a predetermined insertion route for the puncture needle in the ultrasound image.

3. The ultrasound imaging apparatus of claim 1, further comprising a switching unit for switching between activation and deactivation of emphasis of the image of the portion of the puncture needle in the ultrasound image.

4. The ultrasound imaging apparatus of claim 1, further comprising a first setup unit for setting an image lag factor for generating a lag image, the image lag factor for representing movement of the puncture needle, wherein the emphasizing unit generates, by the image lag factor, the lag image in which an image region corresponding to the image of the portion of the puncture needle is emphasized.

5. The ultrasound imaging apparatus of claim 1, further comprising a brightness conversion map selecting unit for selecting a brightness conversion map desired by an operator from a plurality of brightness conversion maps defining a relation between input brightness and output brightness of the ultrasound image, wherein the emphasizing unit adds a predetermined value to brightness of an image region corresponding to the image of the portion of the puncture needle in the ultrasound image and performs brightness conversion with the brightness conversion map selected by the brightness conversion map selecting unit to emphasize the image region corresponding to the image of the portion of the puncture needle.

6. The ultrasound imaging apparatus of claim 5, further comprising a second setup unit for setting an emphasis level of the image of the portion of the puncture needle, wherein the predetermined value added to the brightness of the image region corresponding to the image of the portion of the puncture needle is a value corresponding to the emphasis level set by the second setup unit.

7. The ultrasound imaging apparatus of claim 1, further comprising a brightness conversion map selecting unit for selecting a brightness conversion map desired by an operator from a plurality of brightness conversion maps defining a relation between input brightness and output brightness of the ultrasound image, wherein the emphasizing unit performs gamma conversion on brightness of the ultrasound image with the brightness conversion map selected by the brightness conversion map selecting unit and thereafter adds a predetermined color component to an image region corresponding to the image of the portion of the puncture needle to emphasize the image region corresponding to the image of the portion of the puncture needle.

8. The ultrasound imaging apparatus of claim 1, wherein the portion of the puncture needle is a tip of the puncture needle.

9. An ultrasound image display method, comprising:
generating an ultrasound image based on reception signals output from an ultrasound probe which transmits, in response to drive signals, ultrasound waves to a subject with a puncture needle inserted therein and outputs the reception signals obtained by receiving reflected ultrasound waves from the subject;
selecting a range to emphasize an image of a portion of the puncture needle in the ultrasound image;
detecting the image of the portion of the puncture needle based on a plurality of frames of ultrasound images;
emphasizing the detected image of the portion of the puncture needle within the selected range; and
displaying an ultrasound image in which the image of the portion of the puncture needle is emphasized.

10. The ultrasound image display method of claim 9, wherein the portion of the puncture needle is a tip of the puncture needle.
